(19) United States
(12) Patent Application Publication
(10) Pub. No.: US 2021/0077822 A1
(43) Pub. Date: Mar. 18, 2021

(54) Reconfigurable Cold Plasma Therapy Device with Enhanced Safety

(71) Applicants: Qun Li, Newark, DE (US); Sean Xiaolu Wang, Wilmington, DE (US)

(72) Inventors: Qun Li, Newark, DE (US); Sean Xiaolu Wang, Wilmington, DE (US)

(73) Assignee: BWT Property, Inc., Newark, DE (US)

(21) Appl. No.: 17/004,093

(22) Filed: Aug. 27, 2020

Related U.S. Application Data
(60) Provisional application No. 62/900,700, filed on Sep. 16, 2019.

(51) Int. Cl. A61N 1/44; H05H 1/24

(52) U.S. Cl. CPC A61N 1/44; H05H 2001/2418; H05H 1/2406

(57) ABSTRACT

This invention discloses a reconfigurable cold plasma therapy device with enhanced safety. The plasma therapy device comprises a dielectric barrier discharge (DBD) probe connected to a high voltage power supply, which supplies a high voltage to the DBD probe. The DBD probe comprises a handle having a designated handling area for the operator, whose material and thickness are selected such that when the high voltage is supplied to the DBD probe, the voltage charged onto the body of the operator is below a safety voltage. The low voltage prevents spark generation even when the operator is in close proximity to conductive subjects. As an additional feature, the dielectric barrier of the DBD probe is switchable, allowing both the material and the thickness of the dielectric barrier to be changed to control the property of the plasma discharge for treating different medical conditions.

### REFERENCE TO RELATED APPLICATION

This application claims inventions disclosed in Provisional Patent Application No. 62/900,700, filed Sep. 16, 2019, entitled “RECONFIGURABLE COLD PLASMA THERAPY DEVICE WITH ENHANCED SAFETY.” The benefit under 35 USC § 119(e) of the above mentioned United States Provisional Applications is hereby claimed, and the aforementioned application is hereby incorporated herein by reference.

### FIELD OF THE INVENTION

This invention generally relates to a plasma therapy device, and more specifically to a reconfigurable cold plasma therapy device with enhanced safety.

### BACKGROUND

Plasma as the fourth fundamental state of matter, is a neutral ionized gas composed of positively charged ions, electrons, and neutral particles. In common thermal plasma, all particles approach thermal equilibrium due to intensive collisions between electrons and heavy particles. The temperature in such plasma can reach several thousand degrees. On the other hand, there is another type of plasma in which electrons and heavy particles are in thermal non-equilibrium. In this case, the temperature of the heavy particles is much lower than that of the electrons. This type of plasma is called non-thermal plasma or cold plasma. The heavy particle temperature in cold plasma is typically between 25° C. and 45° C. The plasma discharge may take place in ambient air or in specially supplied gas flow. Many reactive species, including oxygen-based radicals, nitrogen-based radicals, and other components, are generated in the cold plasma. This complicated chemistry can lead to a variety of interactions between cold plasma and biological tissues, allowing the cold plasma to be used for biomedicine.

Dielectric barrier discharge (DBD), which involves electrical discharge between two electrodes separated by an insulating dielectric barrier, is one effective method to produce cold plasma. For biomedical applications, the living tissue is often employed as one of the electrodes, and the plasma discharge is produced between the dielectric barrier and the subject tissue. In general, the electrode and the dielectric barrier are housed in a probe, which connects to a power supply to supply a high voltage in the range from several kV (kilovolt) to a few tens of kV to the electrode. The operator holds the probe to a position close to the subject tissue to produce the plasma discharge. Since the body of the operator is in contact with the probe, it can be charged to a high voltage. As a result, a spark may be ignited when any body part of the operator is in close proximity to a conductive subject. This poses potential health hazards.

### SUMMARY OF THE INVENTION

It is the overall goal of the present invention to solve the above-mentioned problems and provide a reconfigurable cold plasma therapy device with enhanced safety. The plasma therapy device comprises a dielectric barrier discharge (DBD) probe connected to a high voltage power supply, which supplies a high voltage to the DBD probe. The DBD probe comprises a handle having a designated handling area for the operator, whose material and thickness are selected such that when the high voltage is supplied to the DBD probe, the voltage charged onto the body of the operator is below a safety voltage. The low voltage limits/prevents spark generation even when the operator is in close proximity to conductive subjects. As an additional feature, the dielectric barrier of the DBD probe is switchable, allowing both the material and the thickness of the dielectric barrier to be changed to control the property of the plasma discharge for treating different medical conditions.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates one exemplary embodiment of the reconfigurable DBD probe.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a reconfigurable cold plasma therapy device with enhanced safety. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

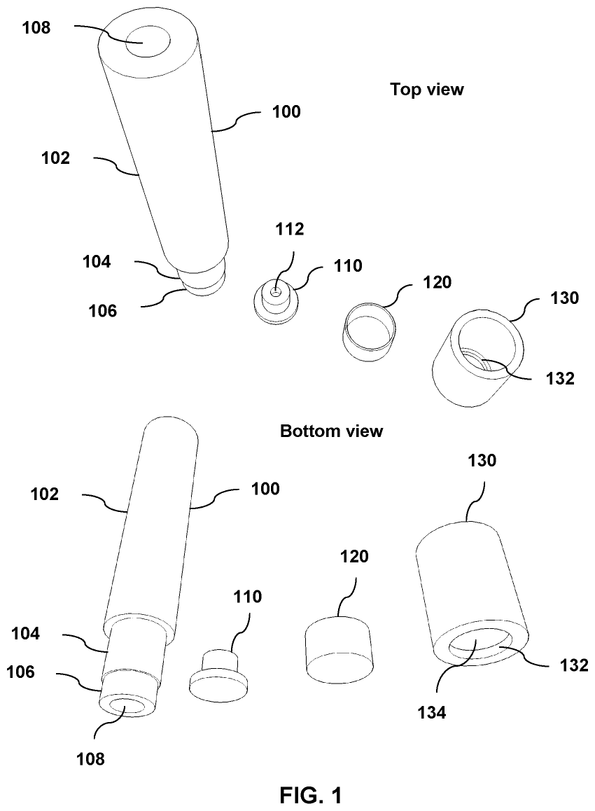

The cold plasma therapy device comprises a dielectric barrier discharge (DBD) probe, as shown in FIG. 1, which is connected to a high voltage power supply through a high voltage cable (both not shown). The power supply is preferably a pulsed power supply with an adjustable repetition rate and output voltage. The output voltage is preferably in the range from 1 kV (kilovolt) to several tens of kV or even higher. The DBD probe further comprises a handle 100, an electrode 110, a close-ended tube 120, and an adaptor 130. The electrode 110 is made of a metal material (e.g., copper) and has a socket 112 on the top to be soldered with the high voltage cable. The electrode 110 and the soldered high voltage cable are mounted into the center hole 108 of the handle 100 from the bottom side. The close-ended tube 120 is then mounted onto the bottom part 106 of the handle to cover the electrode 110. The adaptor 130, which has an opening 134 and a step 132 at its bottom, is employed to hold the close-ended tube 120. The inside of the adaptor 130 and the outside of the middle part 104 of the handle 100 are threaded so that the adaptor 130 can be screwed onto the handle 100 to secure both the close-ended tube 120 and the electrode 110 onto the handle 100. The bottom of the close-ended tube 120, which serves as the dielectric barrier, exposes to the ambient air through the opening 134 of the adaptor 130. The thickness of the step 132 controls the distance from the dielectric barrier to the subject tissue, i.e., the discharge distance. The size of the opening 134 and the bottom part of the electrode 110 controls the plasma discharger area, i.e., the treatment area. The top part 102 of the handle 100 serves as the designated holding area for the operator.

The handle 100 and the adaptor 130 are all made of dielectric material with low dielectric constant (i.e., relative permittivity), and their thickness are selected such that when the high voltage is supplied to the DBD probe, the voltage charged from the handle 100 onto the body of the operator is less than a safety voltage. The low voltage limits/prevents spark generation even when the operator is in close proximity to highly conductive subjects. The safety voltage is preferably less than 4 kV, which will limit the intensity of any possible spark to a level below winter static discharge. More preferably, the safety voltage is less than 2 kV, which makes the spark barely perceptible. Ideally, the safety voltage should be less than 327 V, which is the minimum breakdown voltage of ambient air at standard atmospheric pressure in accordance to Paschen's law. The material (e.g., polyethylene, polypropylene) for the handle 100 and the adaptor 130 preferably has a low dielectric constant of less than 2.5. The overall height of the electrode 110 is made as small as possible so that it is kept a distance (e.g., 20 mm) away from the hand holding area (i.e., the top part 102) of the handle 100 to avoid producing high voltage on the hand holding area.

As one example, the dielectric constant and the thickness of the handle 100 can be selected/designed following the procedures below. First, the electrical property of the body of the operator is modeled in accordance with a standard human body model (HBM) (e.g., IEC 61000, IEC60601, MIL-STD-883). Second, the capacitance of the handle 100, which is determined by its dielectric constant, thickness, and size of the holding area, is calculated. Third, the voltage applied to the electrode 110 is divided between the capacitor of the handle and the capacitor of the body of the operator (which are connected in series) to obtain the voltage on the body of the operator. The dielectric constant and thickness of the handle shall be selected/designed to make this voltage below the safety voltage, as disclosed above.

The close-ended tube 120, which serves as the dielectric barrier, is exchangeable and can be made of different material & thickness to make the DBD probe reconfigurable. The exchangeable dielectric barrier offers additional freedom for controlling the properties of the plasma discharge as its capacitance affects the discharge voltage, and its dielectric constant affects the streamer intensity, diameter, and density of the plasma discharge. A typical material for the tube 120 includes glass, whose dielectric constant may vary from 3.7 to 10 depending on its composition, or alumina, which has a dielectric constant of 9.8 and can withstand high temperatures. As an additional safety measure, a disc made of flexible dielectric material can be placed at the bottom of the tube 120 to prevent direct discharge from the electrode even when the tube 120 breaks. The bottom thickness of the tube 120 shall be kept to a minimum value (e.g., 0.5 mm) so that the voltage drop from the electrode to the bottom surface of the dielectric barrier can be minimized. This lessens the voltage requirement for the power supply, which also makes the plasma therapy device safer.

In the foregoing specification, specific embodiments of the present invention have been described. However, one of ordinary skill in the art appreciates that various modifications and changes can be made without departing from the scope of the present invention as set forth in the claims below. The numerical values cited in the specific embodiment are illustrative rather than limiting. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims, including any amendments made during the pendency of this application and all equivalents of those claims as issued.

What is claimed is:

1. A dielectric barrier discharge (DBD) cold plasma therapy device for treating a subject, the DBD cold plasma therapy device comprising:
a handle having a designated handling area for an operator;
an electrode and a dielectric barrier mounted to the handle, wherein the electrode is enclosed by the dielectric barrier and the handle; and
a high voltage power supply for supplying a high voltage to the electrode to produce a cold plasma discharge between the dielectric barrier and the subject for treating the subject;
wherein the voltage charged from the handle onto the body of the operator is below a safety voltage.

2. The DBD cold plasma therapy device of claim 1, wherein the material and the physical dimension of the handle are controlled to control the voltage charged from the handle onto the body of the operator.

3. The DBD cold plasma therapy device of claim 1, wherein the handle is made of a dielectric material having a low dielectric constant of <2.5.

4. The DBD cold plasma therapy device of claim 1, wherein the electrode is kept a distance away from the handling area.

5. The DBD cold plasma therapy device of claim 4, wherein the distance is >20 mm.

6. The DBD cold plasma therapy device of claim 1, wherein the safety voltage is less than 4 kilovolt (kV).

7. The DBD cold plasma therapy device of claim 1, wherein the safety voltage is less than 2 kilovolt (kV).

8. The DBD cold plasma therapy device of claim 1, wherein the safety voltage is less than 327 volt.

9. The DBD cold plasma therapy device of claim 1, wherein the dielectric barrier is switchable within a set of dielectric barriers having different physical dimensions.

10. The DBD cold plasma therapy device of claim 1, wherein the dielectric barrier is switchable within a set of dielectric barriers made of different materials.

11. The DBD cold plasma therapy device of claim 1, wherein the high voltage power supply is a pulsed, high voltage power supply with adjustable output voltage and repetition rate.
